(19) United States
(12) Patent Application Publication
(10) Pub. No.: US 2019/0159859 A1
(43) Pub. Date: May 30, 2019

(54) OPERATIVE ASSISTANCE SYSTEM FOR A MAGNETIC RESONANCE TOMOGRAPH

(71) Applicant: OTTO-VON-GUERICKE-UNIVERSITÄT MAGDEBURG, TTZ PATENTWESEN, Magdeburg (DE)

(72) Inventors: Enrico PANNICKE, Magdeburg (DE); Klemens OPFERMANN, Magdeburg (DE)

(73) Assignee: Otto-von-Guericke-Universität Magdeburg, TTZ Patentwesen, Mageburg (DE)

(21) Appl. No.: 16/097,716

(22) PCT Filed: May 3, 2017
(86) PCT No.: PCT/DE2017/100367

§ 371 Date: Oct. 30, 2018

(30) Foreign Application Priority Data
May 7, 2016 (DE) 10 2016 005 436.0
May 7, 2016 (DE) 10 2016 005 437.9

(51) Int. Cl. A61B 90/13; G01R 33/28; G01R 33/48; A61B 5/055

(52) U.S. Cl. CPC A61B 90/13; A61B 5/055; G01R 33/48; G01R 33/28

(57) ABSTRACT

The invention relates to an operative assistance system (1) for a magnetic resonance tomograph (I) for displaying an entry point (IX) for an instrument on the surface of a patient (VIII), comprising at least one device (3) having at least one first holder (IV) for accommodating at least one conductor loop (III), and having a second holder (V) for accommodating at least one luminous means (II) for displaying predefinable coordinates of the entry point (IX)).

The invention relates to an operative assistance system for a magnetic resonance tomograph for displaying an entry point for an instrument on the surface of a patient.

Minimally-invasive treatment methods attempt to reduce the stress for the patient, inter alia, in that the patient is injured as little as possible. However, the direct visual monitoring during a treatment is thus also dispensed with. It is therefore necessary to accompany such a treatment by way of an imaging method. Magnetic resonance tomography is particularly suitable for this purpose, since it supplies a good soft tissue contrast and the layers to be displayed can be oriented as desired. The foundation of the image generation is the hydrogen in the human body. Materials which do not contain hydrogen in a suitable form cannot be displayed using this method. This relates above all to the instruments, which can only be visualized by the effect thereof in the patient during the treatment, typically the displacement of tissue.

For an optimum course of treatment it is also necessary for a specific entry point to be selected for the instrument. This can be dependent on a variety of criteria and is planned beforehand by the physician on the basis of diagnostic image data. Finding the previously planned entry point on the patient requires a high degree of imagination on the part of the physician. They have to orient themselves on the surface of the patient on the basis of the two-dimensional layer images of the interior. It would thus be desirable to assist the physician in this task by providing a suitable device, so that the puncture site can be located more rapidly.

Various methods have been presented in the past to find the puncture site for an instrument on the patient.

Fingertip Method

In this method, the plane having the puncture site is recorded continuously and the physician moves their finger over the patient. In this case, they are assisted by an MTA (medical technician) in the scanner vestibule, who monitors the imaging. The advantage of this method is its simple and efficient implementation. No additional tools are necessary to carry it out. However, the physician requires a large amount of experience in orienting themselves in arbitrary layer orientations. Moreover, the efficiency of the method is strongly dependent on the interaction of the MTA with the physician. Short-term changes in the team can cause significant efficiency losses in this case.

Tracking Systems

Tracking systems can be used to track an instrument independently of the imaging. These can then assume important tasks as an “independent” monitoring authority. Present and target coordinates can be compared to one another without problems, however, a visualization accessible to the physician is only possible with difficulty. Such a tracking system is also not available everywhere and can always only monitor a small volume. In addition, the instrument cannot be tracked and therefore a correction cannot be supplied as to how far away it is from the puncture site.

Marker

Instruments can be monitored in an image-based manner by manipulating the properties thereof in the image. This method can be very efficient. However, the rough orientation and position thereof has to be known for this purpose, so that the layers to be recorded can be aligned appropriately.

Planning Software

Unambiguously determinable landmarks are used on the basis of a detailed plan to mark the puncture site. This can take place, for example, in that the location on the z axis is known and the corresponding arc length is measured on the patient. This method has the disadvantage that the treatment scenario cannot deviate from the plan.

DE 102011006650 A1 discloses a device and a method for treating a prostate in a patient, comprising an imaging device for generating an image of the prostate, comprising a treatment needle, which is guided freehand and is introducible through a puncture point on the perineum of the patient into the patient, comprising a navigation system coupling the imaging device and the treatment needle, and comprising a guide means for displaying a setpoint trajectory leading to one of the destinations for the treatment needle, wherein for at least two of the destinations, the respective setpoint trajectories are selected such that they lead to the same entry point.

A magnetic resonance tomograph (MRT) comprising a laser guiding system for directly displaying location and access path of interior-body structures on the patient by means of laser beam technology is known from DE 20000107 U1, wherein the user can localize the target structure using a computer system connected thereto and the software thereof, plan the access path, and move the laser in a computer-controlled manner by means of the diverse movable guide rails, arms, and joints into the position for projection of the planned trajectory.

A magnetic resonance tomograph and a method for displaying a puncture site or marking on the body of a patient for a biopsy or a therapy application during an MRT examination are known from DE 102005046077 A1. The marking on the patient for finding the puncture site is performed by means of a laser, wherein the positioning unit, which is arranged in front of or in the tomograph, has at least one motor for moving the laser along a guide and wherein the motor consists of nonmagnetic parts and is designed such that it does not generate a separate magnetic field during the motor operation.

The known methods each have external drives for controlling the positioning unit.

Against this background, it would be desirable to have an operative assistance system for a magnetic resonance tomograph for displaying an entry point for an instrument on the surface of the patient, which is easy to handle without drastically stressing or even injuring the patient at the same time.

This object is achieved by a device as claimed in claim 1 or 5 and a method as claimed in claim 10. Further advantageous embodiments are disclosed in the dependent claims.

An operative assistance system (1) for a magnetic resonance tomograph (I) for displaying an entry point (IX) for an instrument on the surface of a patient (XII) is proposed, at least comprising:
a rotatably mounted unit (3), at least comprising
a first holder (IV) for accommodating at least one conductor loop (III) and
a second holder (V) for accommodating at least one light source (II),
a light source (II) for displaying predefinable coordinates of the entry point (IX), wherein the unit (3) is arranged adjacent to a lateral surface (I_{M}) of the magnetic resonance tomograph (I),
a mount (VI), and
a control unit (SE) for changing the position and/or alignment of the light source (II),
   wherein the control unit (SE) is electrically connected to the conductor loop (III) and the light source (II) and the first holder (IV) interacts by means of force transmission with the second holder (V) such that the position of the light source (II) is variable.

This construction can be embodied very compactly and nonetheless enables a great variability in the alignment of the light source. The use of the conductor loops also has the great advantage that the arrangement always finds a stable operating point.

If a conductor loop or coil is energized in the inhomogeneous magnetic field of the MRT, a force dependent on the magnetic field thus acts thereon because of the moving charge carriers. This results in a change of the alignment due to a torque and an acceleration of the object. The field strength is decisive for the first effect, while the second effect is dependent on the gradient of the field. It is thus to be presumed in front of the MRT, in the region of the magnetic stray field, that the acceleration effect dominates, while the object changes its alignment in the homogeneous field in the interior of the MRT. In the inhomogeneous field, the acceleration takes place along the positive field change, i.e., toward the field strength becoming greater.

In addition to an electrical connection or transmission of signals between the control unit (SE), the conductor loop (III), and the light source (II), an optical transmission of the signals is also possible.

In one embodiment of the invention, it is provided that the assistance system (1) is arranged on an inner wall of the lateral surface (I_{M}) of the magnetic resonance tomograph (I).

The advantage exists there that it interferes little during the treatment and cannot shine in the eyes of the treating physician.

The assistance system (1) advantageously comprises two conductor loops (III), which are arranged perpendicularly to one another.

This has the result that the resulting dipole moment results as a vector addition of the torques of the conductor loops, i.e., very simple regulation of the deflection is possible via the ratio of the currents in the two conductor loops.

In one embodiment of the invention, the assistance system (1) is characterized in that the unit (3) is mounted so it is rotatable in an angle φ on the mount (VI).

According to the invention, the operative assistance system is located in the homogeneous magnetic field of the magnetic resonance tomograph (I). The placement preferably takes place on the inner side of the tomograph tube (I). For this purpose, at least two conductor loops (III) are fastened on a holder (IV) provided for this purpose. The holders (IV) are mounted by means of the mount (VI) such that they can carry out a rotational movement. The two conductor loops (III) are oriented perpendicularly or orthogonally to one another. The ratio of the currents (VII) flowing in the conductor loops (III) determines the resulting angle φ.

The rotatable conductor loops are advantageously mounted so they are smooth running and can be actuated separately with respect to the polarity and current strength thereof.

It is also conceivable to integrate a measuring device which can register the deflection of the arrangement. Deviations induced by the mechanical construction can thus be compensated for in a regulation.

The light source (II) of the assistance system (1) can be formed in this case as:
a diode and/or
a laser and/or
a mirror system and/or
an incandescent lamp having a focusing device.

Other embodiments are also conceivable, which all at least consist of a conductor loop (III), however. Further coils are preferably energized differently. With suitable mounting (VI) and deflection by a gearing (X), a light source (II) can be aligned in such a manner that a target point (IX) on the patient surface (XII) is marked.

According to the method, the operative assistance system (1) can be used to display an entry point (IX) for an instrument in a magnetic resonance tomograph (I).

Furthermore, an operative assistance system (1) is proposed for a magnetic resonance tomograph (I) for displaying or marking an entry point (IX) for an instrument on the surface of a patient (VIII), at least comprising
a movably mounted unit (3a, 3b, 3c), at least comprising
a first holder (IV) for accommodating at least one conductor loop (III) and
a second holder (V) for accommodating at least one light source (II),
a light source (II) for marking or displaying predefinable coordinates of the entry point (IX), wherein the unit (3a, 3b, 3c) is arranged directly in front of the opening of the magnetic resonance tomograph (I),
a mount (VI), and
a control unit (SE) for changing the position of the light source (II),
   wherein the control unit (SE) is electrically connected to the conductor loop (III) and the light source (II) and the first holder (IV) interacts with the second holder (V) by means of force transmission such that the position of the light source (II) is changeable. This has the result that the position of the marking can be changed on the patient surface (XII). The physician is thus assisted, for example, in finding a puncture site.

The invention is based on the force action of energized conductors in a magnetic field. In an inhomogeneous stray field, these conductors experience an acceleration along the gradient of the field. The force action can be used to deflect other devices or units by way of corresponding planning and mounting of the conductor loops.

The movably mounted unit (3a, 3b, 3c) can be guided as a circular ring or as a linearly formed device in a rotational and/or translational manner in the inhomogeneous magnetic field outside the tomograph or magnetic resonance tomograph (MRT), but directly adjoining thereon. The operative assistance system per se is thus located outside the homogeneous magnetic field of the magnetic resonance tomograph (I). The placement preferably takes place on the outer side of the tomograph tubes (XI).

In addition to an electrical connection or transmission of signals between the control unit (SE), the conductor loop (III), and the light source (II), an optical transmission of the signals is also possible.

One embodiment of the invention provides that the first holder (IV) in the unit (3a) is at least formed curved and one or more conductor loops (III) having power connections (VII) are arranged on the first holder (IV), wherein the conductor loops (III) are separately drivable by means of current and a force acts in the stray field of the magnetic resonance tomograph (I) and the first holder (IV) interacts by means of force transmission with the second holder (V) such that the holder (IV) is rotatable about a longitudinal axis L of the magnetic resonance tomograph (I) and the coordinates of the entry point (IX) for the instrument are markable on the surface of the patient (VIII).

The light source (II) is advantageously rigidly connected to the first holder (IV) via the holder (V).

Furthermore, it is provided that the first holder (IV) is at least formed linear in the unit (3b) and at least one conductor loop (III) having power connections (VII) is arranged on the first holder (IV), wherein the conductor loop (III) is drivable by means of current and a force acts and the first holder (IV) interacts by means of force transmission with the second holder (V) such that the first holder (IV) is movable translationally or linearly in the arrow direction T, such that the second holder (V) is rotatable in the arrow direction R and the coordinates of the entry point (IX) for the instrument are markable on the surface of the patient (VIII). This embodiment can also be formed with any other geometry, in addition to the linear formation of the first holder (IV) in the unit (3b).

The first holder (IV) is advantageously formed as a toothed rack.

In a further embodiment of the invention, it is provided that the assistance system has a cable pull, which interacts by means of force transmission with the first holder (IV) and the second holder (V) such that the alignment of the second holder (V) is changeable in the arrow direction T or R and coordinates of the entry point (IX) are markable on the surface of a patient (VIII) by means of the light source (II).

The light source (II) of the assistance system (1) can be formed in this case as a diode and/or a laser and/or a mirror system and/or an incandescent lamp having a focusing unit.

Other embodiments are also conceivable, which all comprise at least one coil or conductor loop (III), however. Further coils are preferably energized differently. With suitable mounting (VI) and deflection of the force by a gearing (X), the light source II can be aligned in such a manner that the target point (IX) is marked on the patient surface (XII).

According to the method, the operative assistance system (1) for displaying an entry point (IX) for an instrument in a magnetic resonance tomograph (I) can be used for the purpose of processing predefined coordinates (for example, conversion into different reference systems) and moving the unit (3a, 3b, 3c) accordingly.

The invention will be explained in greater detail hereafter on the basis of figures, but it is not restricted thereto.

In the figures:

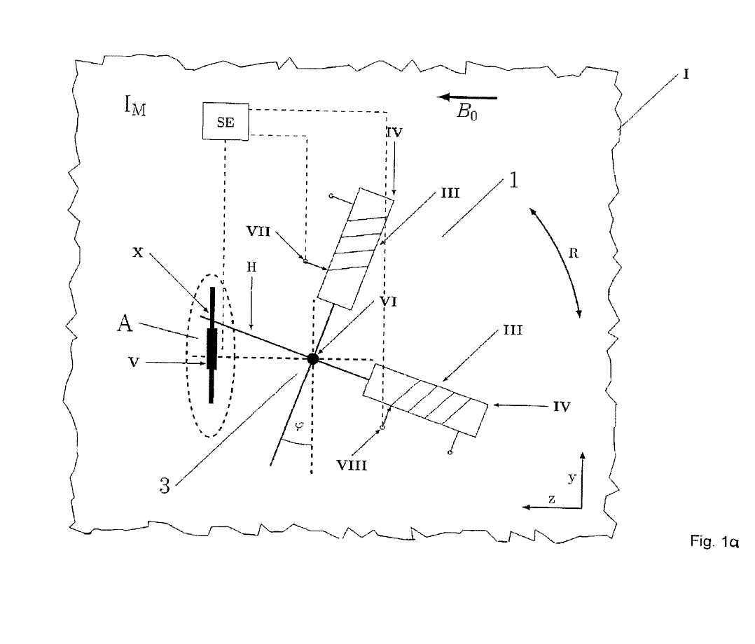

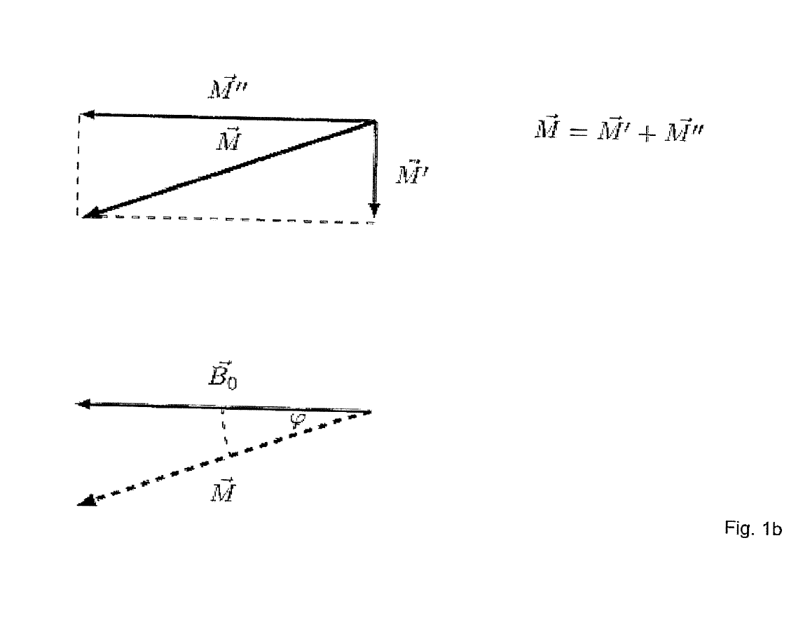

FIGS. 1a and 1b: schematically show an operative assistance system for a magnetic resonance tomograph (MRT) and a vector addition of the dipole moments,

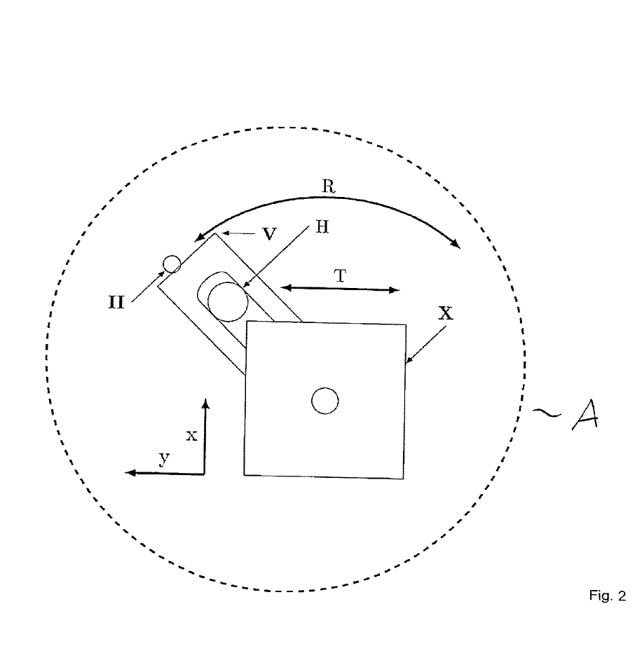

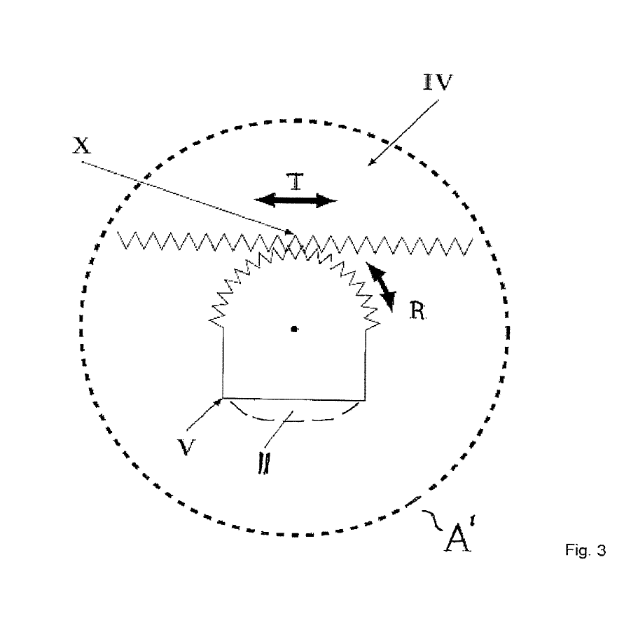

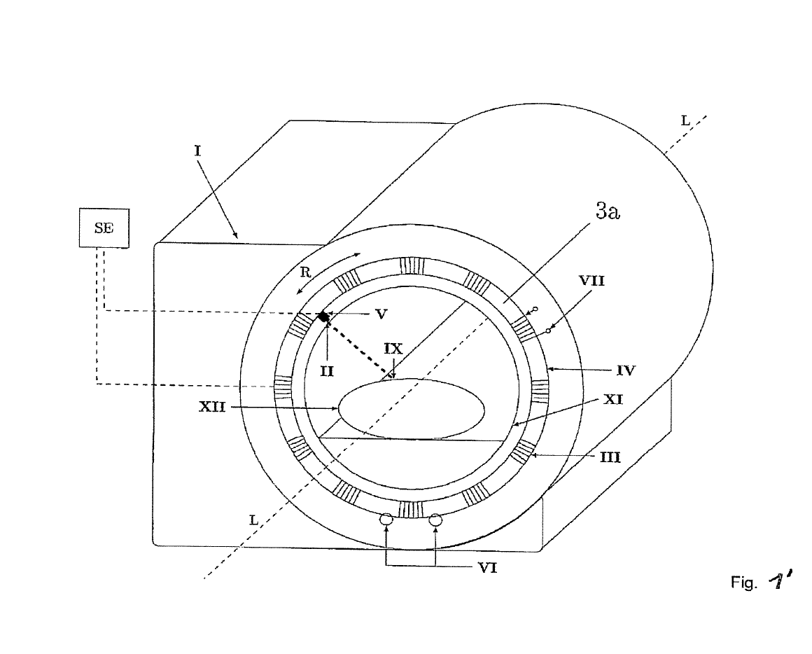

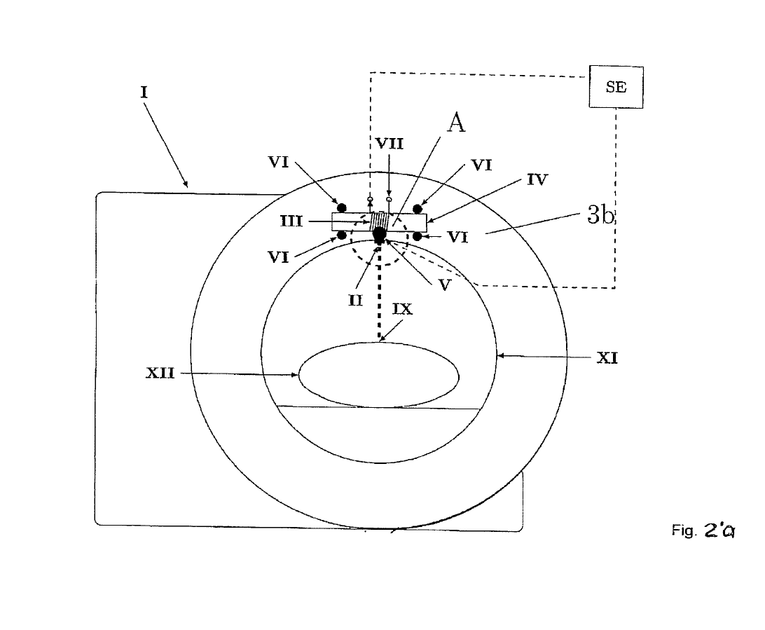

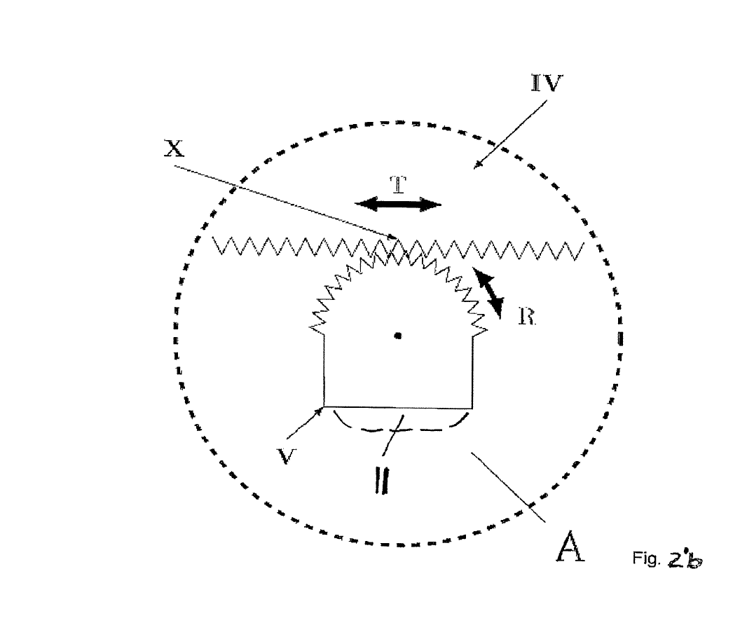

FIG. 2: schematically shows a detail (A) of a gearing (X) of the operative assistance system from FIG. 1, and

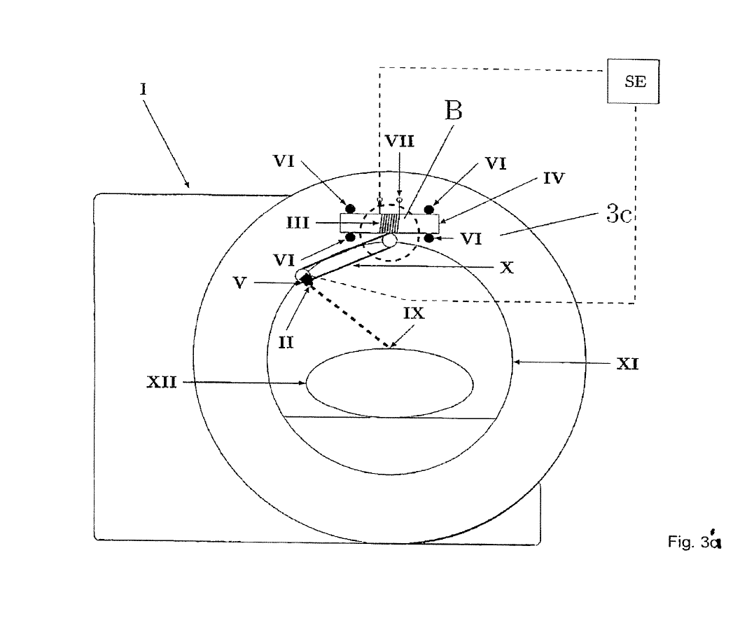

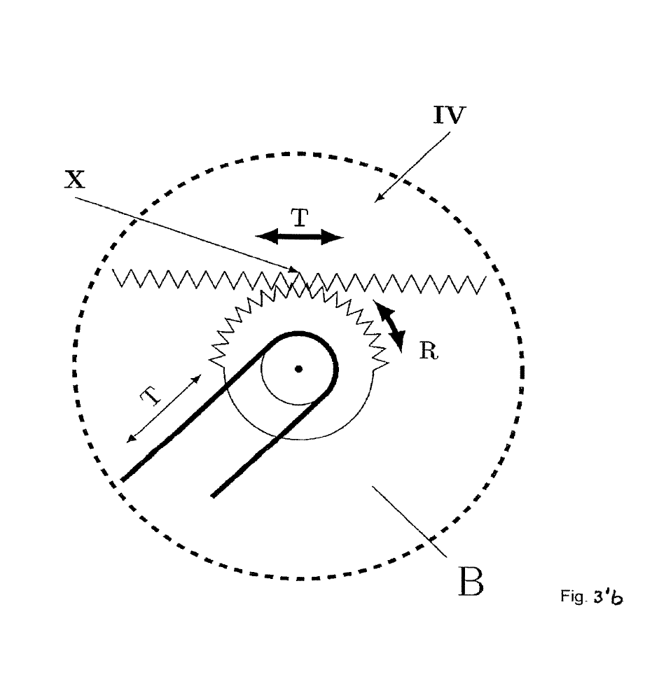

FIG. 3: schematically shows a detail (A′) of the deflection of the light source II.

FIG. 1′: schematically shows an operative assistance system for a magnetic resonance tomograph (MRT) and

FIGS. 2′a and 2′b: schematically show a further embodiment of an operative assistance system for a magnetic resonance tomograph having a detail (A) of a gearing (X) of the operative assistance system and

FIGS. 3′a and 3′b: schematically show a further embodiment of an operative assistance system for a magnetic resonance tomograph having a detail (B) of a gearing (X) of the operative assistance system.

FIGS. 1a and 1b schematically show an operative assistance system for a magnetic resonance tomograph for displaying an entry point (IX) for an instrument on the surface of a patient (XII), wherein the vector addition of the dipole moments is illustrated, which is for the alignment of the arrangement of the positioning system and/or the positioning unit.

In this case, a rotatably mounted unit (3) is provided, which has a first holder (IV) for accommodating at least one conductor loop (III) and a second holder (V) for accommodating at least one light source (II) according to the details (A) and (A′) of FIGS. 2 and 3.

The light source (II) is used in this case for the display of predefinable coordinates of the entry point (IX), wherein the unit (3) is arranged adjacent to a lateral surface (I_{M}) of the magnetic resonance tomograph (I). The assistance system has a mount (VI) and a control unit (SE) for changing the position (alignment) of the light source (II), wherein the control unit (SE) is electrically connected to the conductor loop (III) and the light source (II) and the first holder (IV) interacts by means of force transmission via at least one unit in the form of a third holder (H) with the second holder (V) such that the position of the light source (II) is changeable. The holder (H) can be designed in this case as a lever or a circular disk.

The assistance system (1) is arranged on an inner wall of the lateral surface (I_{M}) of the magnetic resonance tomograph (I) in the coordinate plane “y” and “z”. It comprises, for example, two conductor loops (III), which are arranged perpendicularly to one another. In this case, the unit (3) is mounted on the mount (VI) so it is rotatable in an angle φ.

The conductor loop can be manufactured, for example, from an enameled copper wire having a diameter of 0.15 mm and can have approximately 100 turns. If two conductor loops are used, they are preferably aligned perpendicularly and/or orthogonally to one another. The conductor loops are mounted so they are smooth running and can be driven separately with respect to the polarity and current strength thereof, for example, using a current of 100 mA. The operative assistance system is preferably manufactured from MR-compatible materials.

If a conductor loop or coil is energized in the homogeneous magnetic field of the MRT by means of flowing currents (VII, VIII), a magnetic field thus arises around this coil in dependence on the flowing current. This magnetic field interacts with the static B₀ field of the MRT or tomograph. In the homogeneous magnetic field, this has the result that a torque acts on the conductor loop, which attempts to align the magnetic dipole moment thereof parallel to the B₀ field.

If two conductor loops or coils are energized, the resulting force action is thus mathematically determinable as follows via the vector addition of the two individual dipole moments of the coils:

\vec{M}=\vec{M}′+\vec{M}″

The positioning of a light source can thus take place via the different energizing of the coils. Arbitrary angles φ are settable in this way.

Because the magnetic field of the magnetic resonance tomograph is used by energized conductor loops, external drives, such as piezo drives or pneumatic drives, are not necessary to change the position of the light source (II). The first holder (IV) is capable in combination with a gearing (X), via the second holder (V), of aligning the light source (II) in the rotational direction R.

The light source (II) of the assistance system (1) is designed in this case as a diode and/or a laser and/or a mirror system and/or an incandescent lamp having focusing unit.

FIG. 2 schematically shows the detail (A) of the gearing (X) of the operative assistance system from FIG. 1 in the coordinate plane “y” and “x”, wherein the interaction of the gearing (X) with the third holder (H) is shown by way of example and schematically. An arrow (T) identifies the “planar” force action in this case.

It is recognizable in this case that the holder (H) is connected to the holder (V) in such a manner that the deflection (R direction) of the holder (H), also visible in FIG. 1, results in a force action on (V), i.e., a movement in the T direction. Due to the embodiment of the gearing (X), the force action is converted into a rotation (R), which has the result that the light source (II) may be moved on a circular arc in the R direction.

A deflection of the light source (II) can also take place by way of example via the arrangement according to detail (A′) in FIG. 3.

The mount (IV) is connected to the holder (H) in such a manner that its rotation (R) results in a displacement in the direction (T). The gearing (X) can be formed, for example, as a toothed rack and transfers the displacement (T) into a rotation (R) of the holder (V), on which the light source (II) can preferably be fastened.

FIG. 1′ schematically shows an operative assistance system for a magnetic resonance tomograph for displaying an entry point (IX) for an instrument on the surface of a patient (XII).

In this case, a rotatably mounted unit (3a) is provided, which has a first holder (IV) for accommodating at least one conductor loop (III) and a second holder (V) for accommodating at least one light source (II). The light source (II) is used in this case for marking and/or displaying predefinable coordinates of the entry point (IX), wherein the unit (3a) is arranged directly in front of the opening of the magnetic resonance tomograph (I). The assistance system has a mount (VI) and a control unit (SE) for changing the position of the light source and/or the alignment of the light source (II), wherein the control unit (SE) is electrically connected to the conductor loop (III) and the light source (II) and the first holder (IV) interacts by means of force transmission with the second holder (V) such that the position of the light source (II) is changeable.

This arrangement is located in immediate proximity in front of a magnetic resonance tomograph I. The holder IV for the conductor loops III in FIG. 1′ is embodied as an arc, which can carry out a rotational movement about the longitudinal axis L of the magnetic resonance tomograph I by means of a suitable mount VI. A plurality of conductor loops III, which can be activated separately, are fastened on the holder IV. If a current VII flows through a conductor loop III, a force thus acts thereon in the stray field of the tomograph I, which causes the holder IV to rotate. The light source II is connected to the arc using a rigid holder V. Various target points IX on the patient XII can be marked by a rotation of the arc.

The movably mounted unit (3a) can be formed as a circular ring and can be rotationally guided in the inhomogeneous magnetic field outside the tomograph or magnetic resonance tomograph (MRT), but directly adjoining thereon. The operative assistance system per se is thus located outside the homogeneous magnetic field of the magnetic resonance tomograph (I). The placement preferably takes place on the outer side of the tomograph tubes (XI).

However, the first holder (IV) in the unit (3a) is at least formed curved, wherein one or more conductor loops (III) having power connections (VII) are arranged thereon on the first holder (IV), wherein the conductor loops (III) are separately drivable by means of current and a force acts in the stray field of the magnetic resonance tomograph (I) and the first holder (IV) interacts by means of force transmission with the second holder (V) such that the holder (IV) is rotatable about a longitudinal axis L of the magnetic resonance tomograph (I) and the coordinates of the entry point (IX) for the instrument are markable on the surface of the patient (VIII).

In this case, the light source (II) is rigidly connected via the holder (V) to the first holder (IV).

The conductor loop (III) can be manufactured, for example, from an enameled copper wire having a diameter of 0.15 mm and can have approximately 100 turns. The conductor loops can be energized separately with respect to the polarity and current strength thereof, for example, using a current of 100 mA. The operative assistance system is preferably manufactured from MR-compatible materials.

The light source (II) of the assistance system (1) is formed in this case as a diode and/or a laser and/or a mirror system and/or an incandescent lamp having a focusing unit.

FIG. 2′a schematically shows a further embodiment of an operative assistance system for a magnetic resonance tomograph with respect to the holder (IV) of the conductor loop (II) and FIG. 2′b schematically shows a detail A of a gearing (X) of the operative assistance system (1).

In this case, the first holder (IV) in the unit (3b) is at least formed linear and at least one conductor loop (III) having power connections (VII) is arranged on the first holder (IV), wherein the conductor loop (III) is drivable by means of current and a force acts and the first holder (IV) interacts by means of force transmission with the second holder (V) such that the first holder (IV) is movable translationally (linearly) in the arrow direction T, such that the second holder (V) is rotatable in the arrow direction R and the coordinates of the entry point (IX) for the instrument are markable on the surface of the patient (VIII). In addition, this embodiment can also be formed having any other geometry in addition to the linear formation of the first holder (IV) in the unit (3b).

The first holder (IV) is advantageously formed as a toothed rack.

In this embodiment according to FIG. 2′a, the holder (IV) is formed as a linear guide. If a current (VII) is conducted through the conductor loop (III), a force arises which linearly guides the coil holder in combination with the mount. A translational movement in the T direction to the left or right can thus be generated in dependence on the polarity of the current (VII). The linear movement is converted by the gearing (X) in the form of the toothed rack into a rotation of the holder (V) of the light source (II). This rotation enables the light source (II) to be aligned such that various target points (IX) on the patient (XII) can be marked, as shown in FIG. 2′b. The interaction of a gearing (X) with the first holder (IV) and the second holder (V) is shown by the detail (A) in FIG. 2′b.

The interaction of a gearing (X) with the first holder (IV) and the second holder (V) is shown by the detail (A) according to FIG. 2′b as a portion from FIG. 2′a.

FIGS. 3′a and 3′b schematically show a further embodiment of an operative assistance system for a magnetic resonance tomograph having a detail (B) of a gearing (X) of the operative assistance system.

In this embodiment of the invention in FIG. 3′a, it is provided that the assistance system has a cable pull, which interacts by means of force transmission with the first holder (IV) and the second holder (V) such that the alignment of the second holder (V) is changeable in the arrow direction T or R and coordinates of the entry point (IX) are markable on the surface of a patient (VIII) by means of the light source (II).

In this case, the light source (II) can also be placed at another point.

The force transmission between the holder of the conductor loop (IV) and the holder of the light source (V) is performed here by a gearing (X), which is embodied in FIGS. 3′a and 3′b as a cable pull, wherein the detail (B) of the gearing (X) is shown in FIG. 3′b.

It is also conceivable to place the conductor loop (III) at another point of the tomograph (I). However, it has to be noted that the force action thus changes. In the stray field, an acceleration will occur, and in the homogeneous magnetic field of the interior, a rotation of the object will occur. The correct selection of the gearing (X) is important, which ensures a suitable force transmission between the holder (IV) of the conductor loop (III) and the holder (V) of the light source (II).

The movably mounted unit (3b, 3c) can be guided as a linearly formed unit rotationally and/or translationally in the inhomogeneous magnetic field outside the tomograph or magnetic resonance tomograph (MRT) but directly adjoining thereon. The operative assistance system per se is thus located outside the homogeneous magnetic field of the magnetic resonance tomograph (I). The placement is preferably performed on the outer side of the tomograph tubes (XI).

Because the magnetic field of the magnetic resonance tomograph is used by energized conductor loops (III), external drives, for example, piezo drives or pneumatic drives, are not necessary to change the position of the light source (II). The first holder (IV) is capable of executing rotational and/or translational movements in combination with the gearing (X) via the second holder (V) and aligning the light source (II) in the rotational direction R or the translational direction T.

1. An operative assistance system for a magnetic resonance tomograph for displaying an entry point for an instrument on a surface of a patient, comprising:
a rotatably mounted unit comprising
a first holder for accommodating at least one conductor loop,
a second holder for accommodating at least one light source, and
a light source for displaying predefinable coordinates of the entry point,
wherein the rotatably mounted unit is arranged adjacent to a lateral surface of the magnetic resonance tomograph;
a mount; and
a control unit for changing alignment of the light source,
wherein the control unit is electrically connected to the at least one conductor loop,
wherein the light source and the first holder interact by force transmission with the second holder such that the alignment of the light source is changeable.

2. The assistance system as claimed in claim 1,
wherein the assistance system is arranged on an inner wall of the lateral surface of the magnetic resonance tomograph.

3. The assistance system as claimed in claim 1
wherein the at least one conductor loop comprises two conductor loops which are arranged perpendicularly to one another.

4. The assistance system as claimed in claim 1
wherein the rotatably mounted unit is mounted so it is rotatable in an angle φ on the mount.

5. An operative assistance system for a magnetic resonance tomograph for displaying an entry point for an instrument on a surface of a patient, comprising:
a movably mounted unit comprising
a first holder for accommodating at least one conductor loop,
a second holder for accommodating at least one light source, and
a light source for marking predefinable coordinates of the entry point,
wherein the movably mounted unit is arranged directly in front of an opening of the magnetic resonance tomograph;
a mount; and
a control unit for changing alignment of the light source,
wherein the control unit is electrically connected to the conductor loop,
wherein the light source and the first holder interact by force transmission with the second holder such that the alignment of the light source is changeable.

6. The assistance system as claimed in claim 5,
wherein the first holder in the movably mounted unit is curved,
wherein the at least one conductor loop includes one or more conductor loops having power connections, wherein the one or more conductor loops are separately drivable by current,
wherein a force acts in a stray field of the magnetic resonance tomograph, and
wherein the first holder is rotatable about a longitudinal axis of the magnetic resonance tomograph, and
wherein coordinates of the entry point for the instrument are markable on the surface of the patient.

7. The assistance system as claimed in claim 5,
wherein the first holder is linear and the at least one conductor loop has power connections and is arranged on the first holder, wherein the at least one conductor loop is drivable by current, and
wherein the first holder is movable translationally or linearly and the second holder is rotatable, and wherein coordinates of the entry point for the instrument are markable on the surface of the patient.

8. The assistance system as claimed in claim 5 further comprising a cable pull which interacts by force transmission with the first holder and the second holder such that the alignment of the second holder is changeable and wherein coordinates of the entry point are markable on the surface of a patient by the light source.

9. The assistance system as claimed in claim 5 wherein the light source is selected from the group consisting of
a diode,
a laser,
a mirror system, and
an incandescent lamp having a focusing unit.

10. A method for displaying an entry point for an instrument using an operative assistance system for a magnetic resonance tomograph as claimed in claim 1 comprising illuminating the entry point from different alignments with the light source and marking on the surface of the patient with the light source coordinates for the entry point of the instrument.

11. The assistance system as claimed in claim 1 wherein the light source is selected from the group consisting of a diode, a laser, a mirror system, and an incandescent lamp having a focusing unit.
